(19) United States
(12) Patent Application Publication
(10) Pub. No.: US 2009/0036769 A1
(43) Pub. Date: Feb. 5, 2009

(54) SPREAD SPECTRUM ELECTRIC TOMOGRAPHY

(76) Inventor: Mark J. Zdeblick, Portola Valley, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/171,978

(22) Filed: Jul. 11, 2008

Related U.S. Application Data
(60) Provisional application No. 60/949,193, filed on Jul. 11, 2007.

(51) Int. Cl. A61B 5/05

(52) U.S. Cl. 600/424

(57) ABSTRACT

Methods for locating a sensor element in vivo, e.g., during evaluation of tissue motion, such as of a cardiac tissue motion, e.g., heart wall motion, via electric tomography are provided. In the subject methods, an electric field is applied to a subject in a manner such that the sensing element is present in the applied electric field, and a property of, e.g., a change in, the applied electric field sensed by the sensing element is employed to evaluate a patient internal parameter of interest, e.g., to evaluate movement of tissue location, to evaluate a internal device parameter, such as movement thereof, etc. The invention allows for robust noise discrimination, e.g., by employing a spread spectrum applied electric field. Also provided are systems and devices for practicing the subject methods. In addition, innovative data displays and systems for producing the same are provided. The subject methods and devices find use in a variety of different applications, including cardiac resynchronization therapy.

### CROSS-REFERENCE TO RELATED APPLICATIONS

Pursuant to 35 U.S.C. § 119 (e), this application claims priority to U.S. Provisional Application Ser. No. 60/949,193 filed Jul. 11, 2007; the disclosure of which priority application is herein incorporated by reference.

### INTRODUCTION

In a diverse array of applications, sensing internal parameters of a patient is desired, e.g., for diagnostic or therapeutic purposes. Internal parameters that may be sensed in a given application include physiological parameters (e.g., hemodynamic parameters), implanted device parameters (e.g., location, movement), and the evaluation of motion of tissue motion is desirable.

An example of where evaluation of tissue motion is desirable is cardiac resynchronization therapy (CRT), where evaluation of cardiac tissue motion is employed for diagnostic and therapeutic purposes. CRT is an important new medical intervention for patients suffering from heart failure, e.g., congestive heart failure (CHF). When congestive heart failure occurs, symptoms develop due to the heart's inability to function sufficiently. The aim of resynchronization pacing is to induce the interventricular septum and the left ventricular free wall to contract at approximately the same time. Resynchronization therapy seeks to provide a contraction time sequence that will most effectively produce maximal cardiac output with minimal total energy expenditure by the heart.

### SUMMARY

Methods for locating a sensor element in vivo, e.g., during evaluation of tissue motion, such as of a cardiac tissue motion, e.g., heart wall motion, via electric tomography are provided. In the subject methods, an electric field is applied to a subject in a manner such that the sensing element is present in the applied electric field, and a property of, e.g., a change in, the applied electric field sensed by the sensing element is employed to evaluate a patient internal parameter of interest, e.g., to evaluate movement of tissue location, to evaluate a internal device parameter, such as movement thereof, etc. The invention allows for robust noise discrimination, e.g., by employing a spread spectrum applied electric field. Also provided are devices and systems for practicing the subject methods. In certain embodiments, innovative data processing and display protocols, as well as systems that provided for the same, are provided. The subject methods, devices and systems find use in a variety of different applications, such as cardiac related applications, e.g., cardiac resynchronization therapy, and other applications.

### BRIEF DESCRIPTION OF THE FIGURES

FIG. 1A provides a graphical view of a raw signal obtained at a sensing element according to an embodiment of the invention, while FIG. 1B provides a graphical view of a processed signal according to an embodiment of the invention.

FIG. 2 provides a depiction of various electrical tomography system embodiments of the subject invention.

FIG. 3 provides a view of a system according to a representative embodiment of the invention.

FIG. 4 illustrates an exemplary configuration for electrical tomography, in accordance with an embodiment of the present invention.

FIG. 5 illustrates an exemplary configuration for 3-D electrical tomography, in accordance with an embodiment of the present invention.

FIG. 6 illustrates an electrical tomography system based on an existing pacing system, in accordance with an embodiment of the present invention.

### DETAILED DESCRIPTION

Methods for locating a sensor element in vivo, e.g., during evaluation of tissue motion, such as of a cardiac tissue motion, e.g., heart wall motion, via electric tomography are provided. In the subject methods, an electric field is applied to a subject in a manner such that the sensing element is present in the applied electric field, and a property of, e.g., a change in, the applied electric field sensed by the sensing element is employed to evaluate a patient internal parameter of interest, e.g., to evaluate movement of tissue location, to evaluate a internal device parameter, such as movement thereof, etc. The invention allows for robust noise discrimination, e.g., by employing a spread spectrum applied electric field. Also provided are systems devices for practicing the subject methods. In addition, also disclosed are innovative data processing and display protocols, and systems for performing the same. The subject methods and devices find use in a variety of different applications, e.g., cardiac resynchronization therapy.

In further describing the subject invention, aspects of spread spectrum electrical field tomography methods are reviewed first in greater detail. Next, embodiments of electric field tomography devices and systems are described in greater detail, both generally and in terms of specific embodiments of devices and systems that may be employed in such embodiments. Following this section, embodiments of applications in which the subject invention finds use are described, as well as other aspects of the invention, such as computer related embodiments and kits that find use in practicing the invention.

### Spread Spectrum Electric Tomography Methods

As summarized above, the subject invention provides electric tomography methods for locating a sensor element in vivo, e.g., in evaluating movement of a tissue location of interest. In the subject tomography methods, data obtained by a sensing element, e.g., in motion or stably associated with the tissue location of interest, as it moves through an applied electric field are employed.

Embodiments of the methods may be viewed as “tomography” methods. While the methods may be viewed as tomography methods, such a characterization does not mean that the methods are necessarily employed to obtain a map of a given tissue location, such as a 2-dimensional or 3-dimensional map, but instead just that changes in a sensing element as it moves through an applied electric field are used to evaluate or characterize a tissue location in some way. However, in certain embodiments the data obtained may be processed to obtain and display virtual represent. By “electric field tomography method” is meant a method which employs detected changes in an applied electric field to obtain a signal, which signal is then employed to determine tissue location movement. For the purposes of this application, the term “electric field” means an electric field from which tomography measurement data is obtained. The electric field is one or more cycles of a sine wave. There is no necessary requirement for discontinuity in the field to obtain data. As such, the applied field employed in embodiments of the subject invention is continuous over a given period of time.

The “electric field” used for tomography measurement may, at times, be provided with disruptions or naturally have some disruptions, and still be considered a “continuous field”. As clarifying examples, pulsing the field to conserve power or mutiplexing between different fields remains within the meaning of “continuous field” for the purposes of the present invention. In contrast, a time-of-flight detection method falls outside of the meaning of “continuous field” for the purposes of the present invention. Accordingly, the continuous field applied in the subject methods is distinguished from “time of flight” applications, in which a duration-limited signal or series of such signals is emitted from a first location and the time required to detect the emitted signal at a second location is employed to obtain desired data. At best, if a series of signals are generated in a time of flight application, the series of signals is discontinuous, and therefore not a continuous field, such as the field employed in the present invention.

The underlying precept among the electric field tomography method is that a source is provided which generates a field ψ. ψ varies throughout the internal anatomical area of interest.

One example of the source field ψ can be expressed in a form:

ψ=A sin(2πft+φ)

where:

f is the frequency,

φ is a phase,

A is the amplitude, and

t is time.

In certain embodiments, the field oscillates as a function of time, and can be described simply an AC field.

In obtaining data from the electric field, A, f or φ is a function of some parameter(s) of interest. Two parameters of interest among the many available parameters are location position and location velocity. When one or more properties of the field, e.g., A, f and/or φ, is sampled at various points, and the measured property is compared to the reference value, electrical tomography data is obtained.

For example, if an electrical field driven by an alternating-current (AC) voltage is present in a tissue region, one may detect an induced voltage on an electrode therein. The frequency of the induced voltage, f′, is the same as the frequency of the electrical field. The amplitude of the induced signal, however, varies with the location of the electrode. Hence, by detecting the induced voltage and by measuring the amplitude of the signal, one can determine the location as well as the velocity of the electrode.

In general, electric field tomography can be based upon measurement of the amplitude, frequency, and phase shift of the induced signal. Further details regarding the underlying operating principles of electrical field tomography are provided in PCT application serial no. PCT/US2005/036035; the disclosure of which is herein incorporated by reference.

The applied electric field employed in the present invention is a spread spectrum applied electric field. Spread-spectrum techniques are methods by which energy generated at one or more discrete frequencies is deliberately spread or distributed in time or frequency domains. The spread spectrum electric field may be one that includes a spreading code component, as developed in greater detail below.

In practicing embodiments of the invention, following implantation of any required elements in a subject (e.g., using known surgical techniques), the first step is to set up or produce, i.e., generate, an electric field in a manner such that the sensing element(s) of interest is present in the generated electric field. In certain embodiments, a single electric field is generated, while in other embodiments a plurality of different electric fields are generated, e.g., two or more, such as three or more, e.g., four or more, six or more, etc., where in certain of these embodiments, the generated electric fields may be substantially orthogonal to one another. Of interest in certain embodiments are multiple electrical fields as described in U.S. patent application Ser. No. 11/562,690; the disclosure of which is herein incorporated by reference.

An electric field can be generated such that the voltages applied to two or more electrodes can be adjusted to synthesize a “virtual electrode,” such that the effective position to which the electric fields return is not coincident with either electrode. For example, if three electrodes are positioned at the vertices of an equilateral triangle, and one of the electrodes is selected as ground, while the other two electrodes are energized at the same voltage, the effective direction of the field will be from the ground electrode to a point halfway between the two positive electrodes. By varying the relative voltages on the positive electrodes, the direction of the field can be “steered” to a direction that falls between the two electrodes. By moving the ground electrode, or by varying the voltage on one, two, or all three electrodes, for example, the direction of an electric field can be “steered” or oriented in any arbitrary direction, e.g. in a direction of motion of interest. In certain embodiments, the electric field(s) can be reoriented at least once over a given period of time. The capacity to change orientation of the electric fields and create distinct electrical fields in each of multiple planes can improve resolution in characterizing intracardiac wall motion.

The precision of the “steering”, or the ability to select the direction of the electric field, can be increased by adding more electrodes (e.g. around a ring external to the body, or on a lead). In one embodiment, a belt with many segmented electrodes can be placed around the chest of a subject. By choosing the appropriate linear combination of voltages on the segments, a relatively flat electric field can be generated in an arbitrary orientation. Several fields of different frequency can be superimposed in the same configuration. In certain embodiments, a single electric field is generated, and in some embodiments, two fields that are substantially orthogonal over a large area can be generated. In certain embodiments a plurality of different electric fields can be generated, e.g., two or more, such as three or more, e.g., four or more, six or more, etc., where in certain of these embodiments, the generated electric fields may be substantially orthogonal to one another. In certain embodiments, electric field are generated as described in U.S. patent application Ser. No. 11/562,690; the disclosure of which is herein incorporated by reference.

In practicing the subject methods, the applied electric field(s) may be applied using any convenient format, e.g., from outside the body, from an internal body site, or a combination thereof, as long as the tissue location(s) of interest resides in the applied electric field. The electric field or fields employed in the subject methods may be produced using any convenient electric field generation element, where in certain embodiments the electric field is set up between a driving electrode and a ground element, e.g., a second electrode, an implanted medical device that can serve as a ground, such as a “can” of an implantable cardiac device (e.g., pacemaker), etc. The electric field generation elements may be implantable such that they generate the electric field from within the body, or the elements may be ones that generate the electric field from locations outside of the body, or a combination thereof. As such, in certain embodiments the applied electric field is applied from an external body location, e.g., from a body surface location. In yet other embodiments, the electric field is generated from an internal site, e.g., from an implanted device (e.g. a pacemaker can), one or more electrodes on a lead, such as a multiplexed electric lead (e.g., as described in U.S. patent application Ser. No. 10/734,490; the disclosure of which is herein incorporated by reference); including a segmented electrode lead (e.g., as described in U.S. patent application Ser. No. 11/793,904; the disclosure of which is herein incorporated by reference).

In certain embodiments, the electric field is a radiofrequency or RF field. As such, in these embodiments, the electric field generation element generates an alternating current electric field, e.g., that comprises an RF field, where the RF field has a frequency ranging from about 1 kHz to about 100 GHz or more, such as from about 10 kHz to about 10 MHz, including from about 25 KHz to about 1 MHz. Aspects of this embodiment of the present invention involve the application of alternating current within the body transmitted between two electrodes with an additional electrode pair being used to record changes in a property, e.g., amplitude, within the applied RF field. Several different frequencies can be used to establish different axes and improve resolution, e.g., by employing either RF energy transmitted from a subcutaneous or cutaneous location, in various planes, or by electrodes, deployed for example on an inter-cardiac lead, which may be simultaneously used for pacing and sensing. Where different frequencies are employed simultaneously, the magnitude of the difference in frequencies will, in certain embodiments, range from about 100 Hz to about 100 KHz, such as from about 5 KHz to about 50 KHz. Amplitude information can be used to derive the position of various sensors relative to the emitters of the alternating current.

In practicing embodiments of the invention, the sensor picks up a signal that is the amplitude of any of the frequencies of the applied electric field, where the signal is related to its proximity to the one electrode or the other, its locational access modulates the amplitude of the signal. So at a first end the signal would be a signal of a certain size and at the opposite end the signal is the opposite phase and the amplitude at a certain phase will be higher at a first location and lower at a second location. And so if you know the phase the amplitude relates to the distance between them. From that the X, Y and Z locations of an object can be determined.

In the present invention, instead of having three different frequencies making up the applied electric field, the applied electric field employs a spread spectrum electric field, e.g., as generated by a spreading code. As such, one spreads the spectrum out in the applied electric field. In certain embodiments, the Electric field energy generated in a particular bandwidth is spread in the frequency domain, resulting in a signal with a wider bandwidth.

Aspects of the invention include generating a spread spectrum electric field using one or more spreading codes. The applied electric field may comprise three different spreading codes, e.g., in embodiments where three different electric fields are applied, e.g., one generated using a separate or distinct spreading code. Alternatively, the same spreading code may be employed to generate a spread spectrum electric field that is employed at different times, e.g., at different times in each of three directions. In certain embodiments, a very low data rate approach is achieved by using a spreading spectrum code while the other two channels are turned off to make a measurement one channel at a time. In certain embodiments, three different spreading codes are employed. For example, first, second and third spread codes, e.g., spread code number one, spread code number two and spread code number three, may be set up. Now the sensing element senses the signal. A blocking amplifier at a higher frequency may be employed to relay the sub bits of each of these spreading codes. Next, by applying the spreading code as a deconvolution algorithm, three separate signals, each of which relates to the x, y, z coefficients, are obtained. So instead of simply frequency coding these channels, one is using a spread spectrum and coding system for each of these three channels.

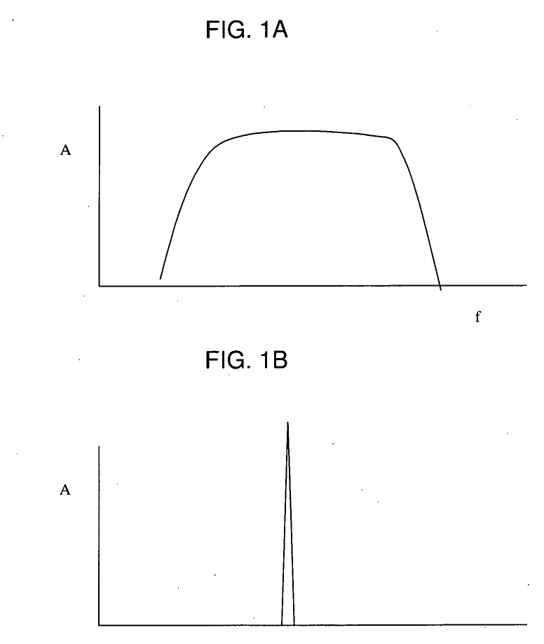

As shown in FIG. 1A, what the spreading approach does is broadcast a signal at a much broader spread spectrum, e.g., by using a pseudo-random code, referring to the graph of panel A. One has a pseudo-random code that has a much broader spectrum and it comes through and then when one deconvolves that, e.g., at the sensing element, it ends up being a much narrower peak with the relevant noise being much smaller, e.g., as illustrated in FIG. 1B. So even if there is interfering noise at various locations, the noise is reduced or even disappears. With spreading technology, one may employ three different codes which basically have the same spectrum but they are distinguishable as three independent peaks, each of which is X, Y and Z. As such, the signal to noise ratio is improved by using three different spread spectrum codes instead of three different frequencies. Spread spectrum electric tomography of the invention finds use in situations where competing noise may be a problem, e.g., where other competing frequencies may be present as be sensed by the sensing element, contributing to noise in the sensed signal.

Any convenient spread spectrum code may be employed to generate the spread spectrum electric field. Spreading spectrum protocols of interest include, but are not limited to: Frequency-hopping spread spectrum (FHSS), direct-sequence spread spectrum (DSSS), time-hopping spread spectrum (THSS), chirp spread spectrum (CSS), and combinations of these techniques. Of interest in certain embodiments is the use of spread spectrum protocols as described in U.S. Pat. Nos. 5,617,871 and 5,381,798; the disclosures of which are herein incorporated by reference. Spread spectrum codes of interest that may be employed in methods of the invention further include those described in: Ziemer and Peterson, Digital Communications and Spread Spectrum Systems (Macmillan Publishing Company, 1985) and Simon et al., Spread Spectrum Communications Handbook (McGraw-Hill Inc., 1994).

In embodiments of the methods, following generation of the applied electric field, as described above, a signal (representing data) from an electric field sensing element that is stably associated with the target tissue location of interest is then detected. In certain embodiments, a signal from the sensing element is detected at least twice over a duration of time, e.g., to determine whether a parameter(s) being sensed by the sensing element has changed or not over the period of time, e.g., to determine whether or not a tissue location of interest has moved over the period of time of interest.

In certain embodiments, a change in a parameter is detected by the sensing element to evaluate movement of the tissue location. In certain embodiments, the detected change may also be referred to as a detected “transformation,” as defined above. Parameters of interest include, but are not limited to: amplitude, phase and frequency of the applied electric field, as reviewed in greater detail below. In certain embodiments, the parameter of interest is detected at the two or more different times in a manner such that one or more of the other of the three parameters is substantially constant, if not constant. In a given embodiment, the sensing element can provide output in an interval fashion or continuous fashion for a given duration of time, as desired.

As summarized above, the subject invention provides methods of evaluating movement of a tissue location. “Evaluating” is used herein to refer to any type of detecting, assessing or analyzing, and may be qualitative or quantitative. In representative embodiments, movement is determined relative to another tissue location, such that the methods are employed to determine movement of two or more tissue locations relative to each other.

The tissue location(s) or site(s) is generally a defined location (i.e. site) or portion of a body, i.e., subject, where in many embodiments it is a defined location or portion (i.e., domain or region) of a body structure, such as an organ, where in representative embodiments the body structure is an internal body structure, such as an internal organ, e.g., heart, kidney, stomach, lung, etc. In representative embodiments, the tissue location is a cardiac location. As such and for ease of further description, the various aspects of the invention are now reviewed in terms of evaluating motion of a cardiac location. The cardiac location may be either endocardial or epicardial, as desired, and may be an atrial or ventricular location. Where the tissue location is a cardiac location, in certain embodiments, the cardiac location is a heart wall location, e.g., a chamber wall, such as a ventricular wall, a septal wall, etc. Although the invention is now further described in terms of cardiac motion evaluation embodiments, the invention is not so limited, the invention being readily adaptable to evaluation of movement of a wide variety of different tissue locations.

By “stably associated with” is meant that the sensing element is substantially if not completely fixed relative to the tissue location of interest, such that when the tissue location of interest moves, the sensing element also moves. As the employed electric field sensing element is stably associated with the tissue location, its movement is at least a proxy for, and in certain embodiments is the same as, the movement of the tissue location to which it is stably associated, such that movement of the sensing element can be used to evaluate movement of the tissue location of interest. The electric field sensing element may be stably associated with the tissue location using any convenient approach, such as by attaching the sensing element to the tissue location by using an attachment element, such as a hook, etc.; by having the sensing element on a structure that compresses the sensing element against the tissue location or is temporarily fixed in position (e.g. a sensing element on a lead or guidewire) such that the two are stably associated; etc. The sensing element may be on a standalone implanted device, or on a carrier, e.g., a lead, guidewire, sheath, etc.

In certain embodiments, a single sensing element is employed. In such methods, evaluation may include monitoring movement of the tissue location over a given period of time. Such embodiments may further include instances where two or more different locations are monitored sequentially, such that a first location is monitored and then the sensing element is moved to a second location which is monitored. For example, a single sensing element may be used to monitor a first location (e.g. an electrode on a cardiac lead at a first location in a cardiac vein) and then the sensing element is moved to a second location which is monitored (e.g. the electrode is placed at a second location in a cardiac vein).

In certain embodiments, two or more distinct sensing elements are employed to evaluate movement of two or more distinct tissue locations. The number of different sensing elements that are employed in a given embodiment may vary greatly, where in certain embodiments the number employed is 2 or more, such as 3 or more, 4 or more, 5 or more, 8 or more, 10 or more, etc. In such multi-sensor embodiments, the methods may include evaluating movement of the two or more distinct locations relative to each other.

The sensing element is, in certain embodiments, an electric potential sensing element, such as an electrode. In these embodiments, the sensing element provides a value for a sensed electric potential which is a function of the location of the sensing element in the generated electric field. In certain embodiments, the electric field sensing element is an electrode. The electrode may be present as a stand alone device, e.g., a small device that wirelessly communicates with a data receiver, or part of a component device, e.g., a medical carrier, such as a lead. Where the sensing element is an electrode on a lead, the lead may be a conventional lead that includes a single electrode. In alternative embodiments, the lead may be a multi-electrode lead that includes two or more different electrodes, where in certain of these embodiments, the lead may be a multiplex lead that has two or more individually addressable electrodes electrically coupled to the same wire or wires. In certain embodiments, a lead, such as a cardiovascular lead, is employed that includes one or more sets of electrode satellites (e.g., that are electrically coupled to at least one elongated conductive member, e.g., an elongated conductive member present in the lead. Multiplex lead structures may include 2 or more satellites, such as 3 or more, 4 or more, 5 or more, 10 or more, 15 or more, 20 or more, etc. as desired, where in certain embodiments multiplex leads have a fewer number of conductive members than satellites. In certain embodiments, the multiplex leads include 3 or less wires, such as only 2 wires or only 1 wire. Multiplex lead structures of interest include those described in application Ser. No. 10/734,490 and U.S. Pat. No. 7,214,189; the disclosures of which application and Patent are herein incorporated by reference.

In certain embodiments, the multiplex lead includes satellite electrodes that are segmented electrodes, in which two or more different individually addressable electrodes are couple to the same satellite controller, e.g., integrated circuit, present on the lead. Segmented electrode structures of interest include, but are not limited to, those described in U.S. Pat. No. 7,214,189 and U.S. patent application Ser. Nos. 11/793,904 and 11/794,016; the disclosures of the various semented multiplex lead structures of these applications being herein incorporated by reference.

In certain embodiments, the subject methods include providing a system that includes: (a) an electric field generation element; and (b) an electric field sensing element that is stably associated with the tissue location of interest. This providing step may include either implanting one or more new elements into a body, or simply employing an already existing implanted system, e.g., a pacing system, for example by using an adapter (for example a module that, when operationally connected to a pre-existing implant, enables the implant to perform the subject methods), as described below. This step, if employed, may be carried out using any convenient protocol.

The subject methods may be used in a variety of different kinds of animals, where the animals are typically “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g., rabbits) and primates (e.g., humans, chimpanzees, and monkeys). In many embodiments, the subjects or patients will be humans.

The subject methods result in the generation of data in the form of signals. From changes determined in these signals obtained from the electric field sensing element, internal parameters of a patient, such as physiological parameters, device parameters, tissue movement, etc., may be determined. for Example, the dynamics and timing of tissue movement can be derived. This rich source of data allows the generation of both physical anatomical dimensions and the physiological functions which they bespeak, typically in real time.

The data obtained using the subject methods may be employed in raw or processed format, as desired and depending on the particular application. In certain embodiments, the obtained data may be processed and displayed to a user, e.g., in the form a computer display, as a graphical user interface (GUI), etc.

The data obtained using the subject methods may be employed in a variety of different applications, including but not limited to, monitoring applications, treatment applications, etc. Applications in which the data obtained from the subject methods finds use are further reviewed in greater detail below.

### Processing of Data

The ET data obtained using the present methods may be employed as raw data or processed in various ways, as desired. For example, using either internal or external orthogonally applied electrical fields, a value for voltage at a tissue location (e.g. an electrode on a cardiac lead, or an epicardial lead) can be obtained to determine a change of voltage. From the voltage data a position signal can be calculated for a location (e.g. an electrode, or a tissue location), and by evaluating the rate of change of the position signal, the position as a function of time can be determined (e.g. the duration of the cardiac cycle). In certain embodiments, at least one of the position signals calculated can be a baseline position signal. In certain embodiments, the position signal can be calculated after an intervention (e.g. a paced position signal, as when employing CRT). In certain embodiments, two or more position signals can be calculated under different conditions (e.g. at baseline, and after pacing with CRT). The position signal(s) can be calculated from a single cardiac cycle, or can be calculated from data averaged over several cardiac cycles, e.g. one cardiac cycle, two cardiac cycles, or three or more cardiac cycles.

The position of a second tissue location (e.g. a second electrode on the same cardiac lead, or an electrode on a separate lead) as a function of time can also be determined by measuring the voltage at that electrode, and the motion at a second tissue location can be compared to motion at a first tissue location. The position of a third, a fourth, a fifth, or more tissue locations (e.g. additional electrodes on the same cardiac lead, or electrodes on a separate lead) as a function of time can also be determined by measuring the voltage at each electrode, and the motion at each tissue location can be compared to motion at other tissue locations.

The position signal can be calculated by separating the monitored voltage data into a cardiac component, an interference component and a noise component. At least one contributor to the interference component is interference from respiration. In some embodiments, calculating the position signal comprises removing the respiration interference component of the measured voltage in order to obtain a position signal. The respiration interference component can be identified and removed in post-processing in order to remove its effect on the position signal generated by cardiac motion. In other embodiments, the respiratory signal can be identified and isolated, and used to compare data sets obtained at the same point in the respiration cycle, usually at end-expiration.

Where desired, the cardiac component data can be normalized, e.g., to increase the accuracy of the position data calculated from the voltage data. Techniques for normalizing the data may include assigning scale factors to signals obtained from a sense electrode to correct for distortions in the electric field. In one embodiment, predetermined scale factors, e.g., based on physiologic characteristics, e.g., the height and weight of the subject, may be employed. In another embodiment, the scale factors can be dynamic, meaning that the scale factors can change over time (e.g. at different points in the cardiac cycle, or from one cardiac cycle to the next) based on changes in the ambient electric fields (e.g. changes in strength, gradient, or direction of the electric field(s) surrounding the sense electrode). In one embodiment, scale factors can be based on a known inter-electrode distance for two or more electrodes that are located in the field, e.g. a one centimeter known separation between two electrodes on a lead, may be employed, where these dimension-based scale factors may be used to correct measurements for the remaining electrodes. In this embodiment, electrodes in close proximity (e.g. 1 cm apart) are electrically coupled. When the lead is bent, the distance between the electrodes decreases thereby changing the electrical coupling. The measured electrical coupling signal provides data related to bending of the lead in the region around the electrodes. This data can be used to normalize signals from the remaining electrodes. A third method involves directly measuring distortion in the electric field to obtain a scale factor, e.g., by using a segmented tetraelectrode as described in U.S. Provisional Application Ser. No. 60/790,507 titled “Tetrahedral Electrode Tomography,” and filed Apr. 7, 2006; the disclosure of which is herein incorporated by reference.

Data processing protocols of interest are further described in U.S. application Ser. Nos. 11/664,340 and 11/731,786, as well as published PCT Application publication no. WO 2006/042039; the disclosures of which are herein incorporated by reference.

### Devices and Systems

In certain embodiments, devices and systems are employed for practicing the ET methods. The system of certain embodiments is made up of the following main components or devices: 1) one or more electrodes with at least one electrode (e.g., the sensing electrode) being stably associated, at least temporarily, with a heart wall, where the heart wall location may be an intracardial or epicardial location, as desired and depending on the particular application; 2) a spread spectrum electric field application element, e.g., that includes a signal generator and receiver (where the signal generator and receiver work together to produce the applied electric field; 3) a signal processor; and 4) a signal display. For CRT applications, in order to optimize CRT in real-time, the electrodes can alternate back and forth between pacing and motion sensing functions.

In certain embodiments, the sense electrode(s) is present on a medical carrier, e.g., lead. Carriers of interest include, but are not limited to, vascular lead structures, where such structures are generally dimensioned to be implantable and are fabricated from a physiologically compatible material. With respect to vascular leads, a variety of different vascular lead configurations may be employed, where the vascular lead in certain embodiments is an elongated tubular, e.g., cylindrical, structure having a proximal and distal end. The proximal end may include a connector element, e.g., an IS-1 or DF-1 connector, for connecting to a control unit, e.g., present in a “can” or analogous device. The lead may include one or more lumens, e.g., for use with a guidewire, for housing one or more conductive elements, e.g., wires, etc. The distal end may include a variety of different features as desired, e.g., a securing means, a particular configuration, e.g., S-bend, etc. In certain embodiments, the elongated conductive member is part of a multiplex lead. Multiplex lead structures may include 2 or more satellites, such as 3 or more, 4 or more, 5 or more, 10 or more, 15 or more, 20 or more, etc. as desired, where in certain embodiments multiplex leads have a fewer number of conductive members than satellites. In certain embodiments, the multiplex leads include 3 or less wires, such as only 2 wires or only 1 wire. Multiplex lead structures of interest include those described in such as a multiplexed electric lead (e.g., as described in U.S. patent application Ser. No. 10/734,490; the disclosure of which is herein incorporated by reference); including a segmented electrode lead (e.g., as described in U.S. patent application Ser. No. 11/793,904; the disclosure of which is herein incorporated by reference). In some embodiments of the invention, the devices and systems may include onboard logic circuitry or a processor, e.g., present in a central control unit, such as a pacemaker can. In these embodiments, the central control unit may be electrically coupled to the lead by one or more of the connector arrangements described above.

This approach can be extended to pacing leads with a plurality of sensing electrodes placed around the heart, which provides a more comprehensive picture of the global and regional mechanical motion of the heart. With multiple electrodes, artifacts such as breathing can be filtered out. Furthermore, multiple electrodes can provide three-dimensional relative or absolute motion information by having electrodes switching between the roles of reference, driver, or sense electrode. A multi-electrode lead, such as a multiplex lead can be used, or multiple electrodes can be present on a guidewire, for example. Indeed any of the electrodes (including a pacemaker can) in this system can be used as a reference, driver, or sense electrode.

This approach can be further extended to employ a variety of electrical field generating elements, creating distinct electrical fields in each of multiple planes, or axes. Sensing electrodes can simultaneously report amplitude from each of the multiplanar electrical fields, thereby improving resolution in characterizing intracardiac wall motion. In one embodiment, three essentially orthogonal fields can be created using internal and/or external field generating elements. For example, the fields can be created with X, Y, and Z axes such that the “X” electric field is oriented in a right/left direction with respect to a patient; the “Y” electric field is oriented in a superior/inferior direction with respect to a patient; and the “Z” electric field is oriented in an anterior/posterior direction with respect to a patient. The three essentially orthogonal fields can also be oriented such that they are aligned with principle axes of the heart, such that a first plane or axis is parallel to the long axis of the left ventricle (“long-axis plane”), a second plane is oriented perpendicular to the first (“short-axis plane”), and a third plane is perpendicular to both the long- and short-axis planes (“four-chamber plane”). Using such resolution-enhancing embodiments can, with proper calibration, yield parameters, including stroke volume and ejection fraction, which are important in CHF management, e.g., as further developed below.

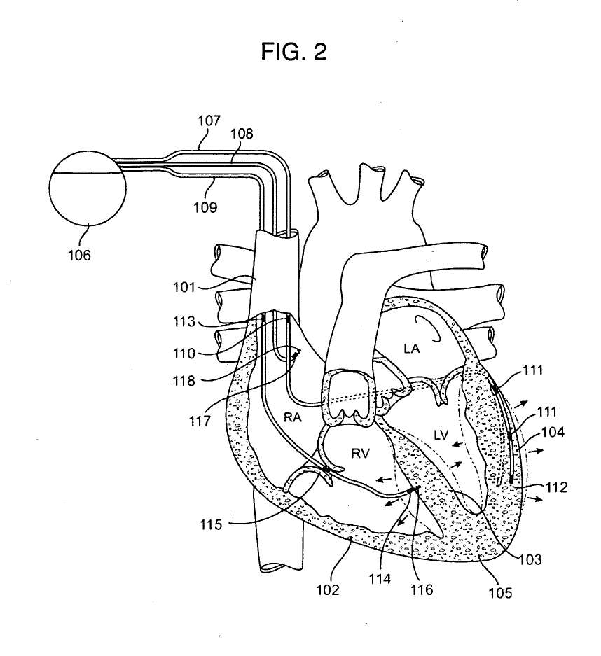

FIG. 2 provides a cross-sectional view of the heart with of an embodiment of the inventive electrical tomographic device, e.g., as embodied in a cardiac timing device, which includes a pacemaker 106, a right ventricle electrode lead 109, a right atrium electrode lead 108, and a left ventricle cardiac vein lead 107. Also shown are the right ventricle lateral wall 102, interventricular septal wall 103, apex of the heart 105, and a cardiac vein on the left ventricle lateral wall 104.

The left ventricle electrode lead 107 is comprised of a lead body and one or more electrodes 110,111, 112. The distal electrodes 111 and 112 are located in a left ventricular cardiac vein and provide regional contractile information about this region of the heart. Also present but not shown are four electrodes in the coronary sinus, in the region of the mitral annulus. The most proximal electrode 110 is located in the superior vena cava in the base of the heart. This basal heart location is essentially unmoving and therefore can be used as one of the fixed reference points for the cardiac wall motion sensing system.

Once the electrode lead 109 is fixed on the septum, electrode lead 109 provides timing data for the regional motion and/or deformation of the septum. The electrode 115 which is located more proximally along electrode lead 109 provides timing data on the regional motions in those areas of the heart. By example, an electrode 115 situated near the AV valve, which spans the right atrium in the right ventricle, provides timing data regarding the closing and opening of the valve. The proximal electrode 113 is located in the superior vena cava in the base of the heart. This basal heart location is essentially unmoving and therefore can be used as one of the fixed reference points for the cardiac wall motion sensing system.

The electrode lead 108 is placed in the right atrium using an active fixation helix 118. The distal tip electrode 118 is used to both provide pacing and motion sensing of the right atrium.

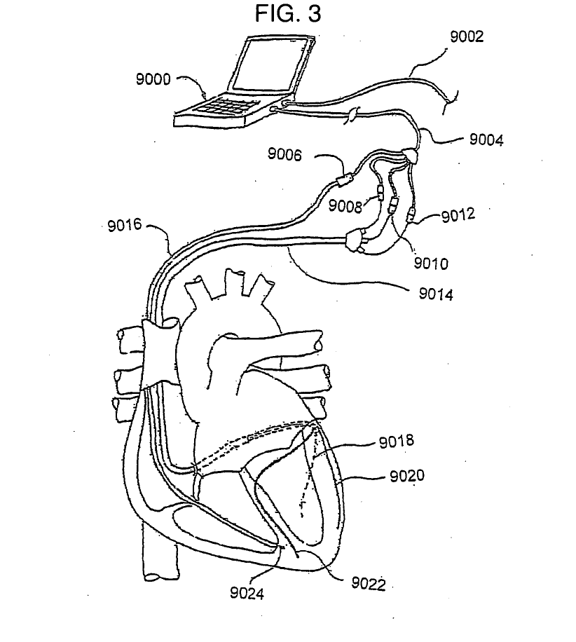

An example of an electrical tomography system according to an embodiment of the present invention is shown in FIG. 3. The embodiment depicted in FIG. 3 is configured to use the electrical tomography technique to measure dysynchronous cardiac motion and assist in optimizing cardiac resynchronization therapy (CRT) for congestive heart failure (CHF) patients as described in this patent application. In FIG. 3, the device is comprised of an electrical tomography system 9000 includes hardware and software for generation of electrical fields, cardiac pacing, data acquisition, data processing, and data display; a skin electrode cable 9002 which is connected to three pairs skin electrodes (right/left torso, chest/back, and neck/leg) which are used to generate three orthogonal electrical fields across the heart; a cardiac electrode cable 9004 which is connected to the internal electrodes within the heart; a guide catheter 9014 which is inserted into the subclavian vein and used to access the coronary sinus; one or more multielectrode guidewires/minicatheters 9018, 9022, and 9024 which have multiple electrodes at the distal end and are inserted via the guidecatheter 9014 into the main cardiac vein and its sidebranches such as the lateral and posterolateral cardiac veins; and a standard RV lead 9024 with an active fixation helical electrode 9024 attached to the septal wall.

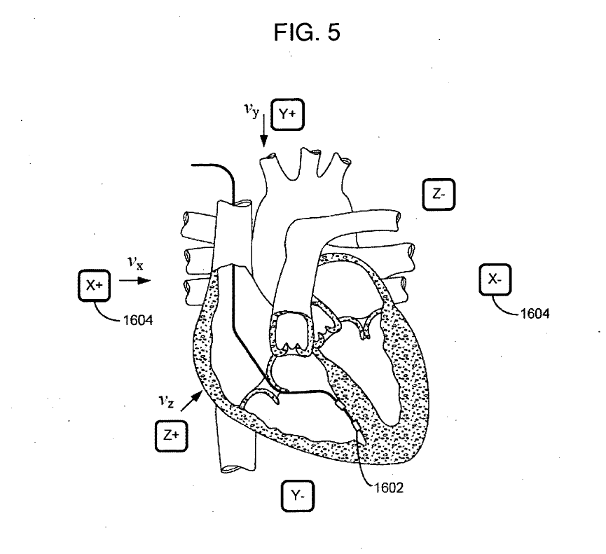

One embodiment of procedural steps would be as follows. The three pairs of skin electrodes are placed on the patient to create the three orthogonal electrical fields spanning the heart. See FIG. 5. The skin electrode cable 9002 is used to connect the skin electrodes to the electrical tomography system 9000. Under sterile field the physician inserts via the subclavian vein an RV lead into the right ventricle and screws the active fixation helical electrode into the septal wall. The physician then uses the guide catheter 9014 to cannulate the coronary sinus. A venogram using a balloon catheter inserted through the guidecatheter 9014 is performed to map the cardiac vein anatomy. The multielectrode guidewires 9018, 9020, 9022 are inserted into the guide catheter 9016. The first multielectrode guidewire 9022 is advanced into the great cardiac vein along the septum until it reaches the apex of the heart. This multielectrode can in addition to the RV electrode lead be used to track the motion of the septal wall. The second multielectrode guidewire 9020 is steered into one of the lateral cardiac veins of the left ventricle. And the third multielectrode guidewire 9018 is steered into one of the postero-lateral cardiac veins of the left ventricle. The cardiac cable 9004 is plugged into the electrical tomography system 9000 and connected to the proximal connectors 9008, 9010, 9012 of the multielectrode guidewires 9018, 9020, 9022, and the proximal IS-1 connector 9006 of the RV electrode lead 9016.

Once all the devices are in place and connected, the three orthogonal electrical fields are turned on and a baseline measurement of the measured motion of all the electrodes is recorded. The amount of baseline intraventricular dyssynchrony is calculated by comparing the motion of the electrodes in the lateral and postero-lateral cardiac veins (multielectrode guidewire 9018, 9020) and the electrodes along the septum (RV lead distal electrode 9024 and/or multielectrode guidewire 9022). Next, CRT test is initiated by performing biventricular pacing with the RV lead distal electrode 9024 and one of the LV electrodes in the lateral or postero-lateral cardiac veins (multielectrode guidewire 9018, 9020). Biventricular pacing is repeated with each of the LV electrodes one by one (multielectrode guidewire 9018, 9020) while recording the corresponding intraventricular dyssynchrony indices. It is important to note that while the LV pacing location is being changed with each test, the motion sensing electrodes used to measure the intraventricular dyssynchrony are not changing position relative to the heart. This allows direct comparison of intraventricular dyssynchrony measurements between all the tests. The data from all the tests is used to generate a map of the optimal LV pacing sites for CRT, thereby identifying the best cardiac vein for placement of the LV electrode lead.

At this point the multielectrode guidewire which is located in the selected cardiac vein is left in place while all the other ones are pulled out. The proximal connector 9008, 9010, or 9012 of the multielectrode lead left in place, is removed and the implantable LV electrode is inserted over-the-wire into the selected cardiac vein and positioned under fluoroscopy to match the position of the determined ideal LV pacing site. In the case of implantation of the multielectrode lead, position within the selected cardiac vein is not critical because of the flexibility provided by the multiple electrodes along the lead.

In another embodiment, at this point all of the multielectrode guidewires are removed and under fluoroscopy the LV electrode lead is positioned using standard lead delivery tools to match the position of the most ideal accessible LV pacing site. Finally, the standard CRT implantation procedure is resumed.

In certain embodiments, a plurality of drive electrode pairs are present, each generating a distinct electric field, where the fields are generally oriented along different endocardial planes, e.g., as may be generated by the different driving electrode pairs shown in FIG. 5. Representative planes generated in certain embodiments are between relatively immobile electrodes located in the superior vena cava, the coronary sinus and an implantable pulse generator in the left or right subclavicular region. Additional electrode locations include the pulmonary artery, and subcutaneous locations throughout the thorax, neck and abdomen, as well as external locations.

In certain embodiments, additional planes are generated from electrodes experiencing relatively greater motion than those already described (e.g., right ventricular apex, cardiac vein overlying left ventricle, etc.). In certain embodiments, to obtain absolute position, computational techniques are employed with reference to other available planes in order to eliminate the motion component of the drive electrodes with respect to the sense electrodes. In certain applications of the system, relative timing and motion information is of greater importance than absolute position. In these applications, at least, significant movement of one or more electrical field planes may be tolerated with minimal or even no real-time computation intended to compensate for this motion.

Another embodiment of the present invention provides a system configured for use in analyzing cardiac motion. During operation, the system places “n” cardiac electrodes and applies an AC voltage to a tissue region where the cardiac electrodes reside. The system then detects an induced voltage on each electrode and constructs an n×n correlation matrix based on the induced voltage on each cardiac electrode. The system subsequently diagonalizes the correlation matrix, thereby solving for eigenvalues and eigenvectors of the correlation matrix.

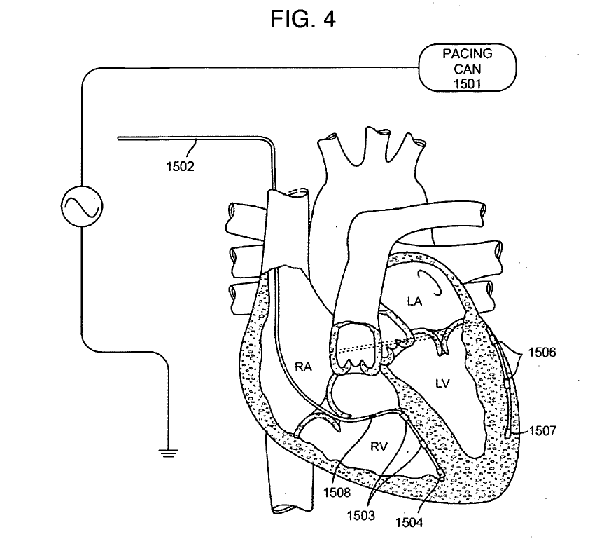

FIG. 4 illustrates an exemplary configuration for electrical tomography of cardiac electrodes, in accordance with an embodiment of the present invention. FIG. 10 shows the locations 1503, 1504, 1506 and 1507 of a number of pacing electrodes. A pacing can 1501 resides in an external or extra-corporeal location. Pacing can 1501 may transmit pacing pulses to the electrodes through a pacing lead 1502.

Electrodes at locations 1503 and 1504 are coupled to right ventricular lead 1502, which travels from a subcutaneous location for a pacing system (such as pacing can 1501) into the patient's body (e.g., preferably, a subclavian venous access), and through the superior vena cava into the right atrium. From the right atrium, right ventricular lead 1502 is threaded through the tricuspid valve to a location along the walls of the right ventricle. The distal portion of right ventricular lead 1502 is preferably located along the intraventricular septum, terminating with fixation in the right ventricular apex. As shown in FIG. 10, right ventricular lead 1502 includes electrodes positioned at locations 1503 and 1504. The number of electrodes in ventricular lead 1502 is not limited, and may be more or less than the number of electrodes shown in FIG. 10.

Similarly, a left ventricular lead follows substantially the same route as right ventricular lead 1502 (e.g., through the subclavian venous access and the superior vena cava into the right atrium). In the right atrium, the left ventricular lead is threaded through the coronary sinus around the posterior wall of the heart in a cardiac vein draining into the coronary sinus. The left ventricular lead is provided laterally along the walls of the left ventricle, which is a likely position to be advantageous for bi-ventricular pacing. FIG. 4 shows electrodes positioned at locations 1506 and 1507 of the left ventricular lead.

Right ventricular lead 1502 may optionally be provided with a pressure sensor 1508 in the right ventricle. A signal multiplexing arrangement facilitates including such active devices (e.g., pressure sensor 1508) to a lead for pacing and signal collection purpose (e.g., right ventricular lead 1502). During operation, pacing can 1501 communicates with each of the satellites at locations 1503, 1504, 1506 and 1507.

According to one embodiment, pacing can 1501 is used as an electrode to apply an AC voltage to the heart tissue. The ground of the AC voltage source may be at another location on the patient's body, for example a patch attached to the patient's skin. Accordingly, there is an AC voltage drop across the heart tissue from pacing can 1501 toward the ground location. An electrode implanted in the heart has an induced electrical potential somewhere between the driving voltage and the ground. By detecting the induced voltage on the electrode, and by comparing the induced voltage with the driving voltage, one can monitor the electrode's location or, if the electrode is moving within the heart, the instant velocity of the electrode. For example, a first signal can be detected at a first time (e.g. the position of an electrode at the beginning of systole), and then at a second time (e.g. the position of the electrode at the end of systole). The velocity can then be computed by differentiating, or taking the derivative of, the position signal of the object (e.g. an electrode). The velocity of an object (e.g. an electrode, or a tissue location) is its speed in a particular direction, or the rate of displacement, and indicates both the speed and direction of an object.

The system may also apply a direct-current (DC) voltage to the tissue. However, an AC driving voltage is preferable to a DC voltage in representative embodiments, because AC signals are more resistant to noise. Because the induced voltage signal on an electrode has substantially the same frequency as the driving AC voltage does, one can use a lock-in amplifier operating at the same frequency to reduce interferences from noise.

The system may apply the electrical field in various ways. In one embodiment, the system may use a pacing can and an existing implanted electrode, or two existing implanted electrodes to apply the driving voltage. In a further embodiment, the system may apply the driving voltage through two electrical-contact patches attached to the patient's skin.

Based on the same principle, one can apply three AC voltages in three directions (x, y, and z), which are substantially orthogonal to each other, to measure the location of an electrode in a 3-dimensional (3-D) space. FIG. 5 illustrates an exemplary configuration for 3-D electrical tomography of cardiac electrodes, in accordance with an embodiment of the present invention. The system applies an AC voltage vₓ through a pair of electrodes 1604 in the x direction. Similarly, the system applies v_{y} and v_{z} in the y direction and z direction, respectively. vₓ, v_{y}, and v_{z} each operates at a different frequency. As a result, three induced voltages are present on an implanted electrode 1602. Each induced voltage also has a different frequency corresponding to the frequency of the driving voltage in each direction. Therefore, by detecting the three induced voltages using three separate lock-in amplification modules, each of which operating at a different frequency, one can determine the electrode's location in a 3-dimensional space.

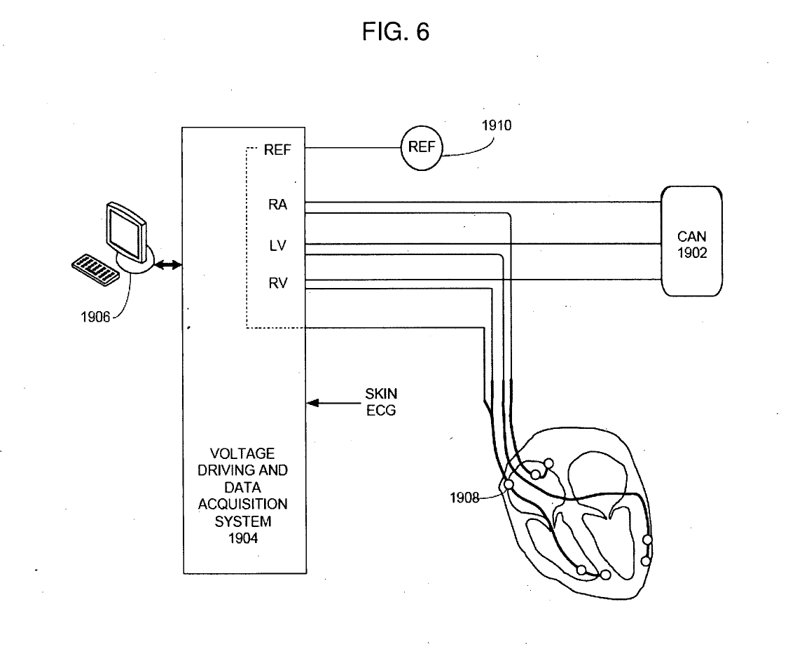

One advantage of an electrode tomography system applying an electrical field is that the system can operate on existing cardiac pacing system and, therefore, incurs minimum risk to a patient. FIG. 6 illustrates an electrical tomography system based on an existing pacing system, in accordance with an embodiment of the present invention. In this example, there are a number of pacing electrodes implanted in a patient's heart. These electrodes may be off-the-shelf electrodes for regular cardiac pacing purposes.

A voltage-driving and data-acquisition system 1904 couples to a pacing can 1902. System 1904 also couples to the electrodes which reside in the right atrium (RA), left ventricle (LV), and right ventricle (RV). Leads from pacing can 1902 are first routed to system 1904 and then routed to the electrodes. System 1904 can use the leads to drive any electrode, including pacing can 1902, and can detect induced signals on non-driving electrodes through the leads. System 1904 also has a reference port which may couple to an external voltage reference point, such as the ground. In the example in FIG. 12, electrode 1908 is coupled through the lead to the reference port, which is coupled to a ground reference voltage 1910.

The arrangement described above allows pacing can 1902 to send regular pacing signals to the electrode while performing electrical tomography. Such simultaneous operation is possible because pacing signals are typically short pulses, whereas the driving voltage is a constant sinusoidal signal with a well defined frequency. Furthermore, system 1904 may receive skin electrocardiogram (ECG) data to assist the analysis of the electrical tomography signals. System 1904 also interfaces with a computer 1906, which performs analysis based on the collected data.

Embodiments of the subject systems incorporate other physiologic sensors in order to improve the clinical utility of wall-motion data provided by the present invention. For example, an integrated pressure sensor could provide a self-optimizing cardiac resynchronization pacing system with an important verification means, since wall motion optimization in the face of declining systemic pressure would be an indication of improper pacing, component failure or other underlying physiologically deleterious condition (e.g., hemorrhagic shock). One or more pressure sensors could also provide important information used in the diagnosis of malignant arrhythmias requiring electrical intervention (e.g., ventricular fibrillation). Incorporation of other sensors is also envisioned.

Effectors of interest include, but are not limited to, those effectors described in the following applications by at least some of the inventors of the present application: U.S. patent application Ser. No. 10/734,490 published as 20040193021 titled: “Method And System For Monitoring And Treating Hemodynamic Parameters”; U.S. patent application Ser. No. 11/219,305 published as 20060058588 titled: “Methods And Apparatus For Tissue Activation And Monitoring”; International Application No. PCT/US2005/046815 titled: “Implantable Addressable Segmented Electrodes”; U.S. patent application Ser. No. 11/324,196 titled “Implantable Accelerometer-Based Cardiac Wall Position Detector”; U.S. patent application Ser. No. 10/764,429, entitled “Method and Apparatus for Enhancing Cardiac Pacing,” U.S. patent application Ser. No. 10/764,127, entitled “Methods and Systems for Measuring Cardiac Parameters,” U.S. patent application Ser. No. 10/764,125, entitled “Method and System for Remote Hemodynamic Monitoring”; International Application No. PCT/US2005/046815 titled: “Implantable Hermetically Sealed Structures”; U.S. application Ser. No. 11/368,259 titled: “Fiberoptic Tissue Motion Sensor”; International Application No. PCT/US2004/041430 titled: “Implantable Pressure Sensors”; U.S. patent application Ser. No. 11/249,152 entitled “Implantable Doppler Tomography System,” and claiming priority to: U.S. Provisional Patent Application No. 60/617,618; International Application Serial No. PCT/USUS05/39535 titled “Cardiac Motion Characterization by Strain Gauge”. These applications are incorporated in their entirety by reference herein.

In the implantable embodiments of this invention, as desired wall motion, pressure and other physiologic data can be recorded by an implantable computer. Such data can be periodically uploaded to computer systems and computer networks, including the Internet, for automated or manual analysis.

Uplink and downlink telemetry capabilities may be provided in a given implantable system to enable communication with either a remotely located external medical device or a more proximal medical device on the patient's body or another multi-chamber monitor/therapy delivery system in the patient's body. The stored physiologic data of the types described above as well as real-time generated physiologic data and non-physiologic data can be transmitted by uplink RF telemetry from the system to the external programmer or other remote medical device in response to a downlink telemetry transmitted interrogation command. The real-time physiologic data typically includes real time sampled signal levels, e.g., intracardiac electrocardiogram amplitude values, and sensor output signals including dimension signals developed in accordance with the invention. The non-physiologic patient data includes currently programmed device operating modes and parameter values, battery condition, device ID, patient ID, implantation dates, device programming history, real time event markers, and the like. In the context of implantable pacemakers and ICDs, such patient data includes programmed sense amplifier sensitivity, pacing or cardioversion pulse amplitude, energy, and pulse width, pacing or cardioversion lead impedance, and accumulated statistics related to device performance, e.g., data related to detected arrhythmia episodes and applied therapies. The multi-chamber monitor/therapy delivery system thus develops a variety of such real-time or stored, physiologic or non-physiologic, data, and such developed data is collectively referred to herein as “patient data”.

### Data Processing

The electrical tomography data obtained using electrical tomography methods and systems, e.g., as described above, may be employed raw or processed as desired, e.g., depending on the particular application which the data is being employed.

In certain embodiments, the data is employed, either alone or in combination with non-ET data (such as data obtained from other types of physiological sensors, e.g., pH sensors, pressure sensors, temperature sensors, etc.) to determine one or more physiological parameters of interest, such as cardiac parameters of interest.

Parameters of cardiac performance measured using this approach can be measured both directly and indirectly. Examples of parameters which can be directly measured include, but are not limited to: cardiac wall motion, including measurements of both intra-ventricular and inter-ventricular synchrony; measurements of myocardial position, velocity, and acceleration in both systole and diastole; measurements of mitral annular position, velocity, and acceleration in both systole and diastole, including peak systolic mitral annular velocity; left ventricular end-diastolic volume and diameter; left ventricular end-systolic volume and diameter; ejection fraction; stroke volume; cardiac output; strain rate; inter-electrode distances; beat-to-beat variation; and QRS duration. Parameters which can be measured indirectly include, but are not limited to: dP/dt (a proxy for contractility); dP/dtₘₐₓ; and calculated measurements of flow including mitral valve flow; mitral regurgitation; stroke volume; and cardiac output. Other parameters which can be measured using the inventive electrical tomography system which are helpful in management of cardiac patients include, but are not limited to: transthoracic impedance, cardiac capture threshold, phrenic nerve capture threshold, temperature, respiratory rate, activity level, hematocrit, heart sounds, sleep apnea determination. In some embodiments, addition sensors (e.g. flow sensors, temperature sensors, pressure sensors, accelerometers, microphone, etc.) may be used to obtain physiologic or cardiac parameters. Both the raw data obtained with this method and processed data can be displayed and used to evaluate cardiac performance.

Parameters which can be measured using the inventive ET system or used in conduction with ET system data include but are not limited to the following:

| | | | Description of |
|---|---|---|---|
| Name | Variable | How Measured | Utility |
| Contractility | dP/dt | Indirect | Change in left ventricular |
| | | Calculate systolic velocity of mitral | pressure over change in |
| | | annulus which correlates with dP/dt | time, Used as a proxy for |
| | | | contractility. |
| | dP/dt_{Max} | Calculate max systolic velocity of mitral | |
| | | annulus which correlates with dP/dt_{Max} | |
| Rate of decline in LV | −dP/dt_{Max} | Indirect (see above) | Used as a proxy for |
| pressure in early diastole | | | contractility |
| End diastolic pressure | EDP | Direct | Gauge Pressure in |
| | | Designated sensor; or | chamber when volume is |
| | | Indirect | maximum |
| | | Measure valve area using ET data, | |
| | | then use formula: | |
| | | ${( \frac{valve area}{{0.11}^{*} SV} )}^{2} = Δ P$ | |
| | | Add ΔP to peripheral diastolic pressure | |
| | | to get ventricular diastolic pressure | |
| End systolic pressure | ESP | Direct | Gauge Pressure in |
| | | Designated sensor; or | chamber when volume is |
| | | Indirect | minimum |
| | | Measure valve area using ET data, | |
| | | then use formula: | |
| | | ${( \frac{valve area}{{0.11}^{*} SV} )}^{2} = Δ P$ | |
| | | Add ΔP to peripheral systolic pressure | |
| | | to get ventricular systolic pressure | |
| Left Ventricular Pressure | LVP | Direct | Gauge pressure in Left |
| | | Designated sensor in LV; or | Ventricle |
| | | Indirect | |
| | | Measure valve area using ET data, | |
| | | then use formula: | |
| | | ${( \frac{valve area}{{0.11}^{*} SV} )}^{2} = Δ P$ | |
| | | Add ΔP to peripheral systolic or | |
| | | diastolic pressure to get ventricular | |
| | | systolic or diatolic pressure | |
| Left Atrial Pressure | LAP | Direct measurement Designated | Reflective of LV filling |
| | | sensor in LA | pressures, which change |
| | | | based upon pump function |
| | | | and fluid status |
| Aortic Pressure | AOP | Direct measurement | Gauge Pressure in aorta |
| | | | just distal to Aortic Valve |
| Pressure Reserve | PR | d(LVESP)/d(LVEDP) | Marginal change in end |
| | | | systolic pressure due to a |
| | | | marginal change in end- |
| | | | diastolic pressure |
| Atrial and Ventricular | | Direct | Volume of cardiac |
| Volumes | | | chambers |
| Left Ventricular End- | LVEDV | Direct | Can track long-term |
| Diastolic Volume | | | evolution of chamber |
| | | | dilation and remodeling |
| Left Ventricular End-Systolic | LVESV | Direct | Can track long-term |
| Volume | | | evolution of chamber |
| | | | dilation and remodeling |
| Left Ventricular Volume | VR | d(LVESV)/d(LVEDV) | Marginal change in end |
| Reserve | | | systolic volume due to a |
| | | | marginal change in end- |
| | | | diastolic volume |
| Atrial and Ventricular | | Direct | Can track long-term |
| Diameters | | | evolution of chamber |
| | | | dilation and remodeling |
| Left ventricular End- | | Direct | Can track long-term |
| Diastolic Diameter | | Use end-diastolic ET position data of | evolution of chamber |
| | | electrodes circumscribing the LV (e.g. | dilation and remodeling |
| | | LV, CS around base of LV and RV | |
| | | apex) to define diameter of LV. | |
| Left Ventricular End-Systolic | | DirectUse end-systolic ET position | Can track long-term |
| Diameter | | data of electrodes circumscribing the | evolution of chamber |
| | | LV (e.g. LV, CS around base of LV and | dilation and remodeling |
| | | RV apex) to define diameter of LV. | |
| Ejection Fraction | EF | Direct | Most commonly used |
| | | (LVEDV-LVESV)/LVEDV | parameter to track LV |
| | | | systolic function. Describes |
| | | | the percentage of blood |
| | | | ejected from a chamber |
| | | | (usually LV) during a cycle. |
| Stroke volume | SV | Direct | Standard cardiac index. |
| | | LVEDV-LVESV | Changes with various |
| | | Corrected for mitral regurgitation if | treatments and |
| | | present (see below) | pathological states. Net |
| | | Indirect | amount of blood ejected |
| | | Measure VTI × CSA = velocity-time | into aorta in one cycle. |
| | | integral of flow at mitral annulus × | |
| | | cross sectional area at mitral annulus | |
| | | Corrected for mitral regurgitation if | |
| | | present (see below) | |
| Stroke Volume Index | SVI | SV/BSA | Stroke volume normalized |
| | | | by Body Surface Area |
| Stroke Reserve | SR | d(SV)/d(LVEDP) | Marginal increase in stroke |
| | | | volume due to a marginal |
| | | | increase in LVEDP |
| Stroke Reserve Index | SRI | d(SVI)/d(LVEDP) | Stroke Reserve normalized |
| | | | by Body Surface Area |
| Stroke Work | SW | ${SV }^{*} ( \overline{{AOP}_{Systole}} - \overline{{LVP}_{Diastole}} )$ | Hemodynamic workperformed by the leftventricle during a singlecycle |
| Stroke Work Index | SWI | SW/BSA | Stroke Work normalized by |
| | | | Body Surfaces Area |
| Stroke Work Reserve | SWR | d(SW)/d(LVEDP) | Marginal increase in Stroke |
| | | | Work due to a marginal |
| | | | increase in LVEDP |
| Stroke Work Reserve Index | SWRI | SWR/BSA | Stroke Work Reserve |
| | | | normalized by Body |
| | | | Surface Area |
| Stroke Power | SP | SW/SEP | Power performed by heart |
| | | | against circulatory system |
| Stroke Power Index | SPI | SP/BSA | Stroke Power normalized |
| | | | by Body Surface Area |
| Stroke Power Reserve | SPR | d(SP)/d(LVEDP) | Marginal Increase Stroke |
| | | | Power due to a marginal |
| | | | increase in LVEDP |
| Stroke Power Reserve | SPRI | SPR/BSA | Stroke Power Reserve |
| Index | | | normalized by body |
| | | | surface areas |
| Cardiac output | CO | Direct or Indirect, depending on | Very commonly used |
| | | method of measuring SV (see above) | cardiac index. Derivative |
| | | SV × HR | of SV. Total amount of |
| | | (stroke volume × heart rate) | blood pumped by the heart |
| | | | per minute. |
| Cardiac Index | CI | CO/BSA | Cardiac output normalized |
| | | | by Body Surface Area |
| Cardiac Reserve | CR | d(CO)/d(LVEDP) | Marginal increase in |
| | | | cardiac output due to a |
| | | | marginal increase in |
| | | | LVEDP |
| Cardiac Reserve Index | CRI | d(CI)/d(LVEDP) | Cardiac Reserve |
| | | | normalized by Body |
| | | | Surface Area |
| Myocardial Work | MyW | ${\int }_{dV \text{/} dt < 0} Pdv - {\int }_{dV \text{/} dt > 0} Pdv$ | Work performed bymyocardial tissue during asingle cycle |
| Myocardial Work Moment | MyWM | ${\int }_{dV \text{/} dt < 0} PVdv - {\int }_{dV \text{/} dt > 0} PVdv$ | Work moment performedby myocardial tissue duringa single cycle |
| Myocardial Work Index | MyWI | MW/BSA | Myocardial work |
| | | | normalized by Body |
| | | | Surface Area |
| Myocardial Reserve | M_{y}R | d(MW)/d(LVEDP) | Marginal increase in |
| | | | myocardial reserve due to |
| | | | a marginal increase in |
| | | | LVEDP |
| Myocardial Reserve Index | M_{y}RI | d(MWI)/d(LVEDP) | Myocardial Reserve |
| | | | normalized by Body |
| | | | Surface Area |
| Myocardial Power | MyP | MyW/SEP | Power performed by the |
| | | | myocardia during systole |
| Myocardial Power Index | MyPI | MyP/BSA | Myocardial Power |
| | | | normalized by body |
| | | | surface area |
| Myocardial Power Reserve | MyPR | d(MyP)/d(LVEDP) | Marginal increase in |
| | | | myocardial power due to a |
| | | | marginal increase in end |
| | | | diastolic pressure |
| Myocardial Power Reserve | MyPRI | MyPR/BSA | Myocardial Power reserve |
| Index | | | normalized by body |
| | | | surface area |
| Myocardial Power | MyPSV | MyP/SV | Power required to deliver |
| Requirement | | | unit stroke volume |
| Cardiac Efficiency | CE | SW/M_{Y}W | Efficiency of the heart in |
| | | | converting myocardial work |
| | | | into circulatory work |
| Cardiac Amplification | CA | d(SV)/d(LVEDV) | Marginal increase in stroke |
| | | | volume due to a marginal |
| | | | increase in LVEDV |
| ET systolic measurements | Sₘ-ET, | Direct | To detect regional wall |
| | e.g. | Measure systolic displacement, | motion abnormalities, a |
| | | velocity, and acceleration data from ET | hallmark of prior infarct (if |
| | | sensing electrodes (e.g., “Sₘ-ET” | unchanged over time) or |
| | | would be the ET correlate of Sₘ, the | ischemia (if dynamically |
| | | maximal velocity of a segment of | changing over time) |
| | | myocardium as measured by TDI) | |
| ET diastolic measurements | Eₐ-ET, e.g. | Direct | Can help to diagnose, |
| | | Measure diastolic displacement, | differentiate and follow |
| | | velocity, and acceleration data from ET | various forms of diastolic |
| | | sensing electrodes. | dysfunction |
| | | “Eₐ-ET” is the ET correlate of Eₐ, the | |
| | | maximal velocity of the MV annulus | |
| | | during early diastolic filling, as | |
| | | measured by TDI) Can be measured | |
| | | from MV annulus (CS) or from other | |
| | | parts of the myocardium. | |
| ET diastolic measurements | | Direct | Can help to diagnose, |
| of LV diastolic filling | | Measured at mitral annulus or left | differentiate and follow |
| Left Ventricular Inflow | | ventricular free wall. | various forms of diastolic |
| Velocities | | | dysfunction. Many of these |
| Early Diastolic Filling | E | Measure early maximum filling velocity | parameters are standard |
| Velocity | | of ventricle after opening of mitral | components of an |
| | | valve. | examination for diastolic |
| Filling Velocity after Atrial | A | Measure second velocity peak in late | dysfunction. |
| Contraction | | diastolic period after atrial contraction. | |
| Ratio of Early Diastolic | E/A | Ratio of E/A | |
| Filling Velocity to Filling | | | |
| Velocity after Atrial | | | |
| Contraction | | | |
| Acceleration/Deceleration | | | |
| Maximal acceleration | | Measure acceleration (derivative of | |
| | | velocity) from time of onset to flow | |
| | | to E velocity. | |
| Early diastolic deceleration | | Measure deceleration from E velocity | |
| slope | | peak to zero baseline. | |
| Myocardial Tissue Velocities | | | |
| Early diastolic myocardial | Eₘ | Measure velocity of myocardium in | |
| tissue velocity | | early diastole. | |
| Diastolic myocardial tissue | Aₘ | Measure velocity of myocardium in | |
| velocity after atrial | | after atrial contraction. | |
| contraction | | | |
| Ratio of early diastolic | Eₘ/Aₘ | Ratio of Eₘ/Aₘ | |
| myocardial | | | |
| tissue velocity and diastolic | | | |
| myocardial tissue velocity | | | |
| after atrial contraction | | | |
| A wave velocity | | Direct | Can help to diagnose, |
| | | Measure small reversal of flow in | differentiate and follow |
| | | atrium following atrial contraction (a | various forms of diastolic |
| | | wave) | dysfunction. |
| E wave velocity | | Direct | Can help to diagnose, |
| | | Measure small reversal of flow at end- | differentiate and follow |
| | | systole (v wave) | various forms of diastolic |
| | | | dysfunction. |
| Propagation Velocity | | Indirect | Measures velocity as blood |
| | | May be estimated from velocity at | moves from mitral annulus |
| | | mitral annulus, LV free wall, or septal | to LV apex |
| | | electrode | |
| Cardiac wall motion | | Direct | To detect regional wall |
| | | Measure ET motion (displacement, | motion abnormalities, a |
| | | velocity, acceleration) data from | hallmark of prior infarct (if |
| | | electrodes on cardiac wall. | unchanged over time) or |
| | | | ischemia (if dynamically |
| | | | changing over time) |
| Intraventricular synchrony | | Direct | Predictor of CRT response; |
| | | Compare timing of ET motion data | assessment of CRT |
| | | from various electrodes around LV. | response |
| Interventricular synchrony | | Direct | Predictor of CRT response; |
| | | Compare timing of ET motion data | assessment of CRT |
| | | from electrodes in LV to pressure | response |
| | | measurement in RV, and/or to timing | |
| | | of electrodes in the RV. | |
| Myocardial position, | | Direct | To detect regional wall |
| velocity, acceleration | | Measure ET motion (position, velocity, | motion abnormalities, a |
| | | acceleration) data from electrodes on | hallmark of prior infarct (if |
| | | cardiac wall, in both systole and | unchanged over time) or |
| | | diastole. | ischemia (if dynamically |
| | | | changing over time) |
| Mitral annular position, | | Direct | Provides important systolic |
| velocity, acceleration | | Calculate velocity and acceleration | and diastolic data. |
| Peak Systolic Mitral | | from ET position data of electrodes in | Systolic velocity correlates |
| Annular Velocity | | coronary sinus (CS) wrapping around | with dP/dtₘₐₓ |
| | | mitral annulus, in both systole and | |
| | | diastole. | |
| Mitral valve flow | | Indirect | MR can change with |
| | | Measure VTI × CSA = velocity-time | pharmacologic and device- |
| | | integral of flow at mitral annulus × | based interventions (e.g., |
| | | cross sectional area at mitral annulus | CRT). |
| Mitral regurgitation | MR | Indirect | MR can respond to |
| | | Measure VTI × CSA = velocity-time | pharmacologic and device- |
| | | integral of retrograde flow at mitral | based interventions (e.g., |
| | | annulus × cross sectional area at mitral | CRT). MR can also come |
| | | annulus | and go with ischemia in |
| | | Indirect | some patients |
| | | Measure cross-sectional area of mitral | |
| | | annulus to infer degree of closure of | |
| | | the leaflets. | |
| Valvular Gradient | VG | ΔPmax | Maximum (during a cycle) |
| | | | pressure gradient across a |
| | | | valve |
| Valvular Gradient Reserve | VGR | d(VG)/d(LVEDP) | Increase in VG as a |
| | | | function of increase in |
| | | | LVEDP. |
| Valvular Area | VA | ${0.11 }^{*} SV \sqrt{Δ P}$ | Standard calculation ofvalvular area using meanpressure gradient andmean flow rate |
| Valvular Area Reserve | VAR | d(VA)/d(LVEDP) | Increase in valvular area |
| | | | as a function of increase in |
| | | | LVEDP |
| Valvular Regurgitation | VR | $\int {Q}_{REGURGITATION}$ | Cumulative regurgitant flowduring a cycle |
| Valvular Regurgitation | VRR | d(VR)/d(LVEDP) | Increase in regurgitant flow |
| Reserve | | | as a function of increase in |
| | | | LVEDP |
| Filling Rates in Atrium and | | Direct | Can help to diagnose, |
| Ventricle | | | differentiate and follow |
| Peak Rapid Filling Rate | | Measure peak velocity and | various forms of diastolic |
| Peak Atrial Filling Rate | | acceleration of ET signals for S, E, | dysfunction. |
| Fractional Filling Rates | | and A waves | |
| Interelectrode distances | | Direct | Measures strain rate, a |
| | | Calculate change in distance of | predictor of CRT response. |
| | | electrodes within close proximity using | |
| | | ET motion data. | |
| Left Ventricular Twist Index | | Direct | Measures degree of |
| | | Measure angular component of | ventricular twisting of apex |
| | | velocity of free wall electrode(s) | with respect to the base |
| Myocardial Strain | | Direct | Predictor of CRT response |
| | | Calculate change in distance of | |
| | | electrodes within close proximity using | |
| | | ET motion data. | |
| Myocardial Strain Rate | SR | Direct | Predictor of CRT response. |
| Max Myocardial Strain Rate | SRₘₐₓ | Calculate rate of change in distance of | |
| | | electrodes within close proximity using | |
| | | ET motion data. | |
| Diastolic Time Intervals | | Direct | Can help to diagnose, |
| Isovolumetric Relaxation | IVRT | Time between aortic valve closure and | differentiate and follow |
| Time | | the onset of ventricular filling (mitral | various forms of diastolic |
| | | valve opening) | dysfunction. |
| Deceleration Time | DT | Time between E peak velocity and | |
| | | zero baseline | |
| Atrial Filling Period | A_{dur} | Measured at mitral annulus | |
| Time from mitral valve | | Time from mitral valve opening to early | |
| opening to E velocity | | maximum diastolic filling velocity | |
| Systolic Time Intervals | | Direct | Can help to diagnose, |
| Systolic Ejection Period | SEP | Time during which blood is ejected | differentiate and follow |
| | | from LV into Aorta | various forms of systolic |
| Time to Onset of Systolic | | Time from beginning of QRS complex | dysfunction, including |
| Velocity | | to beginning of S wave | evaluation of systolic |
| Time to Peak Systolic | Ts | Time from beginning of QRS complex | dyssynchrony. |
| Velocity | | to peak of S wave. | |
| Time to peak acceleration | | Time to maximum systolic acceleration | |
| Time to Peak Post-Systolic | | Time from beginning of QRS complex | |
| Velocity | | to peak post-systolic velocity. | |
| Time to Maximal Systolic | Td | Time from beginning of QRS complex | |
| Displacememt | | to maximum systolic displacement. | |
| Beat to beat variation | R-R | Direct | Can get early warning of |
| | interval | Measure variability of R-R period from | decompensation in heart |
| | | ECG measurements or from ET | failure (HF) and coronary |
| | | electrode motion. | artery disease (CAD) |
| | | | patients. |
| Valve Timing | | Direct | |
| | | Measure impedance change as valve | |
| | | opens | |
| QRS Duration | QRS | Direct | |
| | | Measure length of QRS interval from | |
| | | ECG measurements or from ET | |
| | | electrode motion. | |
| Transthoracic Impedance | | Direct | Can get early warning of |
| | | Thoracic impedance correlates with | decompensation in heart |
| | | fluid status. | failure. |
| Cardiac Capture Threshold | | Direct | To determine lowest |
| | | Measure from EKG or ET electrodes | threshold where cardiac |
| | | | stimulation can be |
| | | | achieved. |
| Heart sounds | | Direct | Measures the timing of |
| | | Microphone or accelerometer in | opening and closing of |
| | | implantable pulse generator. | valves. Helps clarify timing |
| | | | of events in the cardiac |
| | | | cycle. |
| Phrenic Nerve Capture | | Direct | Detect in order to avoid |
| Threshold | | Manifests as sharp spike in ET position | unwanted diaphragmatic |
| | | data. | stimulation |
| Temperature | | Direct | |
| | | Thermocoupling | |
| Respiratory rate | RR | Direct | |
| | | Can detect from impedance data, or | |
| | | signal from ET data | |
| Activity level | | Indirect | |
| | | Accelerometer in implantable pulse | |
| | | generator | |
| Hematocrit | HCT | Direct | |
| | | Blood resistivity | |

As such, a value for a parameter of interest can be obtained from the ET data provided by the methods and systems. The parameter can be one that is derived solely from ET data, or one that is derived from both ET and non-ET data, e.g., data from other types of physiological sensors, e.g., as described above.

### Displaying Data

In certain embodiments, the obtained data is displayed to a user, where the displayed data may be raw data or data that has been processed, e.g., using one or more data processing algorithms. The displayed data may be displayed in any convenient format, e.g., printed onto a substrate, such as paper, provided on a display of a computer monitor, etc. The displays may be in the form of plots, graphs, or any other convenient format, where the formats may be two dimensional, three-dimensional, included data from non-ET sources, etc. Displays of interest include, but are not limited to: those disclosed in PCT application serial no. PCT/US2006/012246 titled “Automated Optimization of Multi-Electrode Pacing for Cardiac Resynchronization,” and filed on Mar. 31, 2006; and U.S. patent application Ser. No. 11/731,78 filed on Mar. 30, 2007; the disclosures of which are herein incorporated by reference.

In certain embodiments, the data is displayed to a user in a graphical user interface. The phrase “graphical user interface” (GUI) is used to refer to a software interface designed to standardize and simplify the use of computer programs, as by using a mouse to manipulate text and images on a display screen featuring icons, windows, and menus. GUIs of interest include, but are not limited to: those disclosed in PCT application serial no. PCT/US2006/012246 titled “Automated Optimization of Multi-Electrode Pacing for Cardiac Resynchronization,” and filed on Mar. 31, 2006 and U.S. patent application Ser. No. 11/731,78 filed on Mar. 30, 2007; the disclosures of which are herein incorporated by reference. GUI displays can be tailored to assist the clinician during clinical situations, such as but not limited to: during implantation of the sensing or pacemaker leads; during initial adjustment of CRT parameters or later “tune-up” of CRT parameters in the clinician's office; and for long-term tracking of cardiac performance.

### Applications

The electric field tomography methods of evaluating tissue location movement find use in a variety of different applications. As indicated above, an important application of the subject invention is for use in cardiac resynchronization, or CRT, also termed biventricular pacing. As is known in the art, CRT remedies the delayed left ventricular mechanics of heart failure patients. In a desynchronized heart, the interventricular septum will often contract ahead of portions of the free wall of the left ventricle. In such a situation, where the time course of ventricular contraction is prolonged, the aggregate amount of work performed by the left ventricle against the intraventricular pressure is substantial. However, the actual work delivered on the body in the form of stroke volume and effective cardiac output is lower than would otherwise be expected. Using the subject tomography approach, the electromechanical delay of the left lateral ventricle can be evaluated and the resultant data employed in CRT, e.g., using the approaches reviewed above and/or known in the art and reviewed at Col. 22, lines 5 to Col. 24, lines 34 of U.S. Pat. No. 6,795,732, the disclosure of which is herein incorporated by reference.

In a fully implantable system the location of the pacing electrodes on multi electrode leads and pacing timing parameters may be continuously optimized by the pacemaker. The pacemaker frequently determines the location and parameters which minimizes intraventricular dyssynchrony, interventricular dyssynchrony, or electromechanical delay of the left ventricle lateral wall in order to optimize CRT. This cardiac wall motion sensing system can also be used during the placement procedure of the cardiac leads in order to optimize CRT. An external controller could be connected to the cardiac leads and a skin patch electrode during placement of the leads. The skin patch acts as the reference electrode until the pacemaker is connected to the leads. In this scenario, for example, the optimal left ventricle cardiac vein location for CRT is determined by acutely measuring intraventricular dyssynchrony.

The subject methods and devices can be used to adjust a resynchronization pacemaker either acutely in an open loop fashion or on a nearly continuous basis in a closed loop fashion.

In certain embodiments, the systems and methods are employed to measure coupling between other electrode locations. The placement and selection of electrode pairs will determine the physical phenomenon that is measured. For instance the voltage coupling between an electrode in the right ventricle and an electrode in the right atrium provides an indication of the timing of the tricuspid valve closing and opening. In certain embodiments, a multiplicity of electrodes on a single lead. For instance a LV pacing lead might have electrodes in addition to the conventional pacing electrodes that extend from the vena cava, through the coronary sinus, and into a cardiac vein on the LV freewall. By selecting different pairs of these electrodes, different aspects of the heart's motion may be measured, as desired.

The subject methods and devices can also be employed in ischemia detection. It is well understood that in the event of acute ischemic events one of the first indications of such ischemia is akinesis, i.e., decreased wall motion of the ischemic tissue as the muscle becomes stiffened. As such, the present methods and devices provide a very sensitive indicator of an ischemic process, by ratiometrically comparing the local wall motion to a global parameter such as pressure. One can derive important information about unmonitored wall segments and their potential ischemia. For example, if an unmonitored section became ischemic, the monitored segment would have to work harder and have relatively greater motion in order to maintain systemic pressure and therefore ratio metric analysis would reveal that fact.

The subject methods and devices also find use in arrhythmia detection applications. Current arrhythmia detection circuits rely on electrical activity within the heart. Such algorithms are therefore susceptible to confusing electrical noise for an arrhythmia. There is also the potential for misidentifying or mischaracterizing arrhythmia based on electrical events when mechanical analysis would reveal a different underlying physiologic process. Accordingly,

Additional applications in which the subject invention finds use include, but are not limited to: the detection of electromechanical dissociation during pacing or arrhythmias, differentiation of hemodynamically significant and insignificant ventricular tachycardias, monitoring of cardiac output, mechanical confirmation of capture or loss of capture for autocapture algorithms, optimization of multi-site pacing for heart failure, rate responsive pacing based on myocardial contractility, detection of syncope, detection or classification of atrial and ventricular tachyarrhythmias, automatic adjustment of sense amplifier sensitivity based on detection of mechanical events, determination of pacemaker mode switching, determining the need for fast and aggressive versus slower and less aggressive anti-tachyarrhythmia therapies, or determining the need to compensate for a weakly beating heart after therapy delivery (where these representative applications are reviewed in greater detail in U.S. Pat. No. 6,795,732, the disclosure of which is herein incorporated by reference), and the like.

In certain embodiments, the subject invention is employed to overcome barriers to advances in the pharmacologic management of CHF, which advances are slowed by the inability to physiologically stratify patients and individually evaluate response to variations in therapy. It is widely accepted that optimal medical therapy for CHF involves the simultaneous administration of several pharmacologic agents. Progress in adding new agents or adjusting the relative doses of existing agents is slowed by the need to rely solely on time-consuming and expensive long-term morbidity and mortality trials. In addition, the presumed homogeneity of clinical trial patient populations may often be erroneous since patients in similar symptomatic categories are often assumed to be physiologically similar. It is desirable to provide implantable systems designed to capture important cardiac performance and patient compliance data so that acute effects of medication regimen variation may be accurately quantified. This may lead to surrogate endpoints valuable in designing improved drug treatment regimens for eventual testing in longer-term randomized morbidity and mortality studies. In addition, quantitative hemodynamic analysis may permit better segregation of drug responders from non-responders thereby allowing therapies with promising effects to be detected, appropriately evaluated and eventually approved for marketing. The present invention allows for the above. In certain embodiments, the present invention is used in conjunction with the a system as described in PCT Application Serial No. PCT/US2006/016370 titled “Pharma-Informatics System” and filed on Apr. 28, 2006; the disclosure of which is herein incorporated by reference.

In certain embodiments, electrodes (e.g. a multi-electrode lead) can be placed in the heart which are connected to the receiver, which can be employed to measure cardiac parameters of interest, e.g., blood temperature, heart rate, blood pressure, movement data, including synchrony data, as well as pharmaceutical therapy compliance. The obtained data is stored in the receiver. Embodiments of this configuration may be employed as an early heart failure diagnostic tool. This configuration may be put into a subject in the early stages of heart failure, with the goal of monitoring them closely and keeping them stable with optimized therapeutic management. Ultimately, when stimulation therapy is required, the receiver may be replaced with an implantable pulse generator, which may then employ the stimulating electrodes to provide appropriate pacing therapy to the subject.

Non-cardiac applications will be readily apparent to the skilled artisan, such as, by example, measuring the congestion in the lungs, determining how much fluid is in the brain, assessing distention of the urinary bladder. Other applications also include assessing variable characteristics of many organs of the body such as the stomach. In that case, after someone has taken a meal, the present invention allows measurement of the stomach to determine that this has occurred. Because of the inherently numeric nature of the data from the present invention, these patients can be automatically stimulated to stop eating, in the case of overeating, or encouraged to eat, in the case of anorexia. The present inventive system can also be employed to measure the fluid fill of a patient's legs to assess edema, or other various clinical applications.

### Computer Readable Storage Media

One or more aspects of the subject invention may be in the form of computer readable media having programming stored thereon for implementing the subject methods. The computer readable media may be, for example, in the form of a computer disk or CD, a floppy disc, a magnetic “hard card”, a server, or any other computer readable media capable of containing data or the like, stored electronically, magnetically, optically or by other means. Accordingly, stored programming embodying steps for carrying-out the subject methods may be transferred or communicated to a processor, e.g., by using a computer network, server, or other interface connection, e.g., the Internet, or other relay means.

More specifically, computer readable medium may include stored programming embodying an algorithm for carrying out the subject methods. Accordingly, such a stored algorithm is configured to, or is otherwise capable of, practicing the subject methods, e.g., by operating an implantable medical device to perform the subject methods. The subject algorithm and associated processor may also be capable of implementing the appropriate adjustment(s).

Of particular interest in certain embodiments are systems loaded with such computer readable mediums such that the systems are configured to practice the subject methods.

### Kits

As summarized above, also provided are kits for use in practicing the subject methods. The kits at least include a computer readable medium, as described above. The computer readable medium may be a component of other devices or systems, or components thereof, in the kit, such as an adaptor module, a pacemaker, etc. The kits and systems may also include a number of optional components that find use with the subject energy sources, including but not limited to, implantation devices, etc.

In certain embodiments of the subject kits, the kits will further include instructions for using the subject devices or elements for obtaining the same (e.g., a website URL directing the user to a webpage which provides the instructions), where these instructions are typically printed on a substrate, which substrate may be one or more of: a package insert, the packaging, reagent containers and the like. In the subject kits, the one or more components are present in the same or different containers, as may be convenient or desirable.

As is evident from the above results and discussion, the subject invention provides numerous advantages. Advantages of various embodiments of the subject invention include, but are not limited to: low power consumption; real time discrimination of multiple lines of position possible (one or more); and noise tolerance, since the indicators are relative and mainly of interest in the time domain. A further advantage of this approach is that there is no need for additional catheters or electrodes for determining position. Rather the existing electrodes already used for pacing and defibrillation can be used to inject AC impulses at one or more frequencies designed not to interfere with the body or pacing apparatus. As such, the subject invention represents a significant contribution to the art.

It is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method for evaluating motion of a tissue site in a subject, said method comprising:
(a) generating a spread spectrum electric field so that a tissue site is present in said electric field;
(b) obtaining an initial signal from a first sense electrode stably associated with said tissue site;
(c) deconvolving said initial signal to obtain a final signal; and
(d) evaluating motion of said tissue site from said final signal.

2. The method according to claim 1, wherein said spread spectrum electric field is generated using a pseudorandom number sequence.

3. The method according to claim 2, wherein said spread spectrum electric field is a Frequency-hopping spread spectrum electric field.

4. The method according to claim 1, wherein said spread spectrum electric field is a direct-sequence spread spectrum electric field.

5. The method according to claim 1, wherein said method comprises generating a single spread spectrum electric field.

6. The method according to claim 1, wherein said method comprises generating two or more spread spectrum electric fields.

7. The method according to claim 6, wherein said two or more spread spectrum electric fields are each generated using a unique spread code.

8. The method according to claim 6, wherein said two or more spread spectrum electric fields are generated using a common spread code.

9. The method according to claim 1, wherein said method comprises generating three spread spectrum electric fields.

10. The method according to claim 9, wherein said method comprises generating three substantially orthogonal spread spectrum electric fields.

11. The method according to claim 1, wherein said initial and final signals are voltage.

12. The method according to claim 1, wherein said method further comprises employing obtaining a final signal from a second sense electrode stably associated with a second tissue site.

13. The method according to claim 1, wherein said evaluating comprises determining a cardiac parameter.

14. The method according to claim 1, wherein said spread spectrum electric field is generated internally.

15. The method according to claim 1, wherein said spread spectrum electric field is generated externally.

16. The method according to claim 1, wherein said sense electrode is present on carrier.

17. The method according to claim 16, wherein said carrier is a lead.

18. The method according to claim 17, wherein said lead comprises a single sense electrode.

19. The method according to claim 17, wherein said lead is a multi-electrode lead.

20. The method according to claim 19, wherein said multi-electrode lead is a multiplex lead.

21. The method according to claim 20, wherein said multi-electrode lead comprises a segmented electrode.

22. A system for evaluating movement of a tissue location, said system comprising:
(a) a spread spectrum electric field generation element;
(b) a sense electrode configured to be stably associated with a cardiac tissue location; and
(c) a signal processing element configured to employ a signal obtained from said sense electrode to evaluate movement of tissue in a method according to claim 1.

23. A computer readable storage medium having a processing program stored thereon, wherein said processing program operates a processor to operate a system comprising:
(a) a spread spectrum electric field generation element;
(b) a sense electrode configured to be stably associated with a cardiac tissue location; and
(c) a signal processing element configured to employ a signal obtained from said sense electrode to evaluate movement of tissue
in a method according to claim 1 to perform a method according to claim 1.
